(19) United States
(12) Patent Application Publication
(10) Pub. No.: US 2017/0150878 A1
(43) Pub. Date: Jun. 1, 2017

(54) LARYNGOSCOPE

(71) Applicant: OBP Corporation, Lawrence, MA (US)

(72) Inventor: Jeffrey Ralph Swift, Boca Grande, FL (US)

(21) Appl. No.: 15/364,754

(22) Filed: Nov. 30, 2016

Related U.S. Application Data
(60) Provisional application No. 62/261,054, filed on Nov. 30, 2015.

(51) Int. Cl. A61B 1/267; A61B 1/00; A61B 1/06

(52) U.S. Cl. CPC A61B 1/267; A61B 1/0684; A61B 1/00027; A61B 1/00103

(57) ABSTRACT

One aspect comprises a laryngoscope assembly with a handle and a blade, the blade including a cavity housing a lighting system, and a cover locked in place over the cavity via a snap-latch, wherein at least the blade is molded from a semi-crystalline polymer. Another aspect comprises a laryngoscope assembly with a handle and a blade extending from the handle, the blade including a cavity, and a lighting system housed within the cavity and including a light source, power source, activation device, and switch, wherein at least the blade is molded from polyarylamide.

### CROSS-REFERENCE TO RELATED APPLICATIONS

This application claims priority to U.S. Provisional Pat. App. No. 62/261,054, filed on Nov. 30, 2015, and entitled “Laryngoscope.” The entire contents of that application are incorporated herein by reference.

### INTRODUCTION

One or more exemplary embodiments described herein relate to a laryngoscope providing a handle, and a blade extending therefrom including an integrated light source, that provides improved vision.

In an exemplary embodiment, the laryngoscope may be suitable for single-use, at which point it may then be discarded

Laryngoscopes are a common surgical tool used by physicians to assist with tracheal intubation of a patient. For example, laryngoscopes may be used following induction of general anesthesia, and during advanced cardiopulmonary resuscitation.

Conventional laryngoscopes include a handle portion containing a light source and a blade portion, with the blade portion including a blade and a light transmission system, such as fiber optic cable. However, in current laryngoscopes, the power supply for the light source is often cumbersome and bulky, and relatively large in size.

Laryngoscopes often require optimal visualization in the visualization area, so that a medical professional can quickly visualize the field of view. For example, it is important for the medical professional to quickly locate the vocal cords and pass the intubation tube through them.

Current laryngoscopes require sterilization after each use, in order to prevent transmission of germs and bacteria, and to attempt to ensure no patient cross contamination. However, laryngoscopes are difficult to sterilize, and some laryngoscope blades with an attached light source cannot be autoclaved (sterilized in a pressure chamber). This can be due to the size of the bulky laryngoscope and attached light source in comparison to the autoclave. Additionally, the sterilization process is not particularly successful with certain microbes, and even after sterilization, the laryngoscope still poses a risk of cross-infection between patients. Moreover, a reused laryngoscope also reduces its functional life.

Therefore, there is a need for an affordable and effective fully disposable, or one time use, laryngoscope. Existing disposable laryngoscopes require a light-source that is removed and reused, while the blade and handle, formed from injection moldable plastic, are discarded. Thus, existing disposable laryngoscopes are not fully disposable, since they require reuse of certain components. Therefore, there still exists the possibility of cross-contamination between patients, due to the inability of sterilization to reduce cross-contamination as a result of component reuse.

Existing disposable laryngoscopes that are fully disposable, including the light and power source, which are integrated into the blade, do exist. However, these laryngoscopes house the light and power source in an enclosure attached to the blade, which impacts the field of view of the laryngoscope. For example, the enclosure obscures and/or blocks visualization of vocal cords during intubation procedures by blocking the field of view. As a result, the success rate of intubation procedures is lowered, and there is an increase in patient risk.

Therefore, there is a need for a fully-disposable laryngoscope blade providing an integrated light source and forming an unobstructed, illuminated view of an area.

One aspect of the invention described herein comprises a laryngoscope assembly comprising: (a) a handle; (b) a blade, the blade including a cavity, the cavity housing a lighting system; and (c) a cover locked in place over the cavity via a snap-latch, wherein at least the blade is molded from a semi-crystalline polymer.

Another aspect comprises a laryngoscope assembly comprising: (a) a handle; (b) a blade extending from the handle, the blade including a cavity; and (c) a lighting system housed within the cavity and including a light source, power source, activation device, and switch, wherein at least the blade is molded from a semi crystalline polymer.

Another aspect comprises a laryngoscope assembly comprising: (a) a handle; (b) a blade extending from the handle, the blade including a cavity; and (c) a lighting system housed within the cavity and including a light source, power source, activation device, and switch, wherein at least the blade is molded from polyarylamide.

In various embodiments of the above and other aspects: (1) the switch further comprises an activation mechanism including an insulating tab that projects outward from the blade, and wherein upon removal of the insulating tab a light source is activated; (2) the cavity is sized such that it tapers in size from a proximal end of the blade to a distal end of the blade; (3) the blade is formed substantially straight, in a style of a Miller blade; (4) the blade is formed substantially curved, in a style of a Macintosh blade; (5) the light source is an LED light source; (6) at least the blade is molded from a low conductivity polymer; (7) at least the blade is molded from a radiolucent polymer; (8) at least the blade is molded from a polymer that is at least 50% glass-fiber reinforced; (9) at least the blade is molded from a polymer that is a polyarylamide compound; (10) at least the blade is molded from a thermoplastic crystalline polymer; (11) at least the blade is molded from a thermoplastic crystalline polymer of aromatic diamines and aromatic dicarboxylic anhydrides; (12) at least the blade is molded from an at least 50% glass-fiber reinforced polyacrylamide; (13) at least the blade is molded from a polymer with a conductivity of less than 10-6 A; (14) at least the blade is molded from a polymer with a flexural modulus of at least 17 Gpa; (15) at least the blade is molded from a polymer with a flexural strength of at least 375 Mpa; (16) at least the blade is molded from a polymer with an impact strength of at least 100 J/M.

Further aspects and embodiments will be apparent from the attached drawings and the detailed description below.

### BRIEF DESCRIPTION OF THE DRAWINGS

FIG.1 is a trimetric view of an exemplary embodiment.

FIG. 2 is a side view of an exemplary embodiment.

FIG. 3 is another side view of an exemplary embodiment.

FIG. 4 is a side view of an exemplary embodiment, with a cover removed.

FIG. 5 is a top view of an exemplary embodiment.

FIG. 6 is a fluoroscopy image illustrating the radiolucency of an embodiment.

FIG. 7 illustrates flexural strength and flexural modulus for a variety of plastics.

### DETAILED DESCRIPTION OF CERTAIN EXEMPLARY EMBODIMENTS

The following description is not to be taken in a limiting sense, but is made merely for the purpose of illustrating certain aspects of a laryngoscope according to the invention.

One or more exemplary embodiments provide a laryngoscope including a handle; a blade extending from the handle; and a light source integral to the laryngoscope blade. The laryngoscope may include tapered lighting and a power enclosure, such that a field of view, looking down the laryngoscope from a proximal end of the blade to a distal end of the blade, is improved. The laryngoscope may be designed for single-use.

The lighting system may be integrated, and built into, the blade portion of the laryngoscope. The lighting system may include a light source, a power source, an activation mechanism and an electrical interconnection system.

The light source may be any suitable light source, including, but not limited to, an LED bulb, halogen bulb, krypton bulb, or xenon bulb. The power source may be any suitable power source, such as, but not limited to, one or more batteries, such as a disposable battery. The activation mechanism may include a pull tab, a switch, and any other suitable activation components. The electrical interconnection system may include a printed circuit board and one or more wires, and may form a circuit.

In an exemplary embodiment, the activation mechanism may include an insulating tab that provides a break in the lighting system circuit. The tab may include an end projecting outward from the laryngoscope. The tab may be removable, and upon pulling and removing the tab, the circuit is completed and the light source is activated.

In an exemplary embodiment, the laryngoscope blade minimizes the number of components for optimal laryngoscope function and design, as compared to conventional laryngoscopes.

In accordance with certain exemplary embodiments, the blade and handle are designed to be compatible with standard high-volume two-part injection molds. The molded blade in accordance with these embodiments provides structural integrity for intubation application, as well as structural support for internal lighting, switching, and power supply components. This provides for minimizing components of the laryngoscope, and compatibility with high-volume injection molding techniques. Accordingly, the laryngoscope of these embodiments is optimized for single-use.

In an exemplary embodiment, the housing for the lighting and power systems of the laryngoscope tapers along the length of the blade portion from the proximal end of the blade to the distal end of the blade. The location of the housing for the lighting, power source, and switch in a proximal end of the blade, which tapers off toward the distal end, provides for an extended field of view with increased visualization.

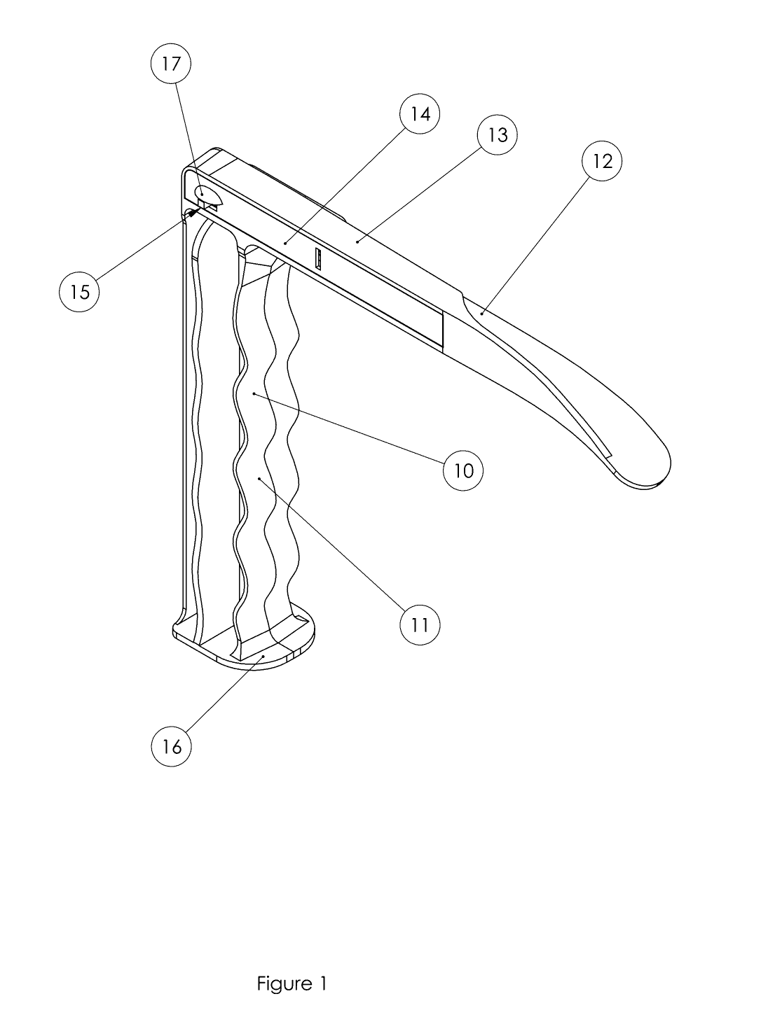

Referring now to FIGS. 1 through 5, an exemplary embodiment may include a single-use disposable laryngoscope 10. The laryngoscope 10 may be formed from hard plastic in a one-piece construction, or any other suitable material. The laryngoscope 10 may be constructed using materials that provide strength to lift up to 15 kilograms or more, and are smooth enough to glide easily over a surface of a human tongue.

The laryngoscope 10 may include a handle 11 and blade 12. Handle 11 and blade may be joined to one other at any suitable angle. For example, blade 12 may be joined to handle 11 at a 75 degree or approximately 75 degree angle. At this angle, the laryngoscope is ergonomically easy to use. In another example, blade 12 may be joined to handle 11 at an approximately 60 degree or approximately 90 degree angle, or any suitable variation thereof.

The dimensions of laryngoscope 10 may be any suitable dimensions. For example, handle 11 may be approximately 12 centimeters in length. In one embodiment, blade 12 is of a greater length than handle 11. In another embodiment, blade 12 may be of a shorter length than, or equal length to, handle 11.

Handle 11 is shaped such that it provides ease-of-use for a user, such as a medical professional, to grasp and use. Handle 11 extends away from blade 12 to form platform 16 at a distal end of the handle, furthest away from blade 12. Platform 16 extends perpendicularly outward from the end of handle 11, and prevents hand slippage during use of the laryngoscope 10.

Laryngoscope 10 includes a housing cavity 13 integrally attached to blade 12. Housing cavity 13 includes the lighting system, which may include one or more of the light source, power source, and switch circuit. The housing cavity 13 includes a switch 15 on a side of the housing cavity for switching the light source on and off. Above switch 15 is a protective extrusion 17, which extends outward from the housing cavity 13 to prevent inadvertent switching of the switch 15 during use of the laryngoscope. The housing cavity 13 is covered by a cover 14, which prevents access to the lighting system.

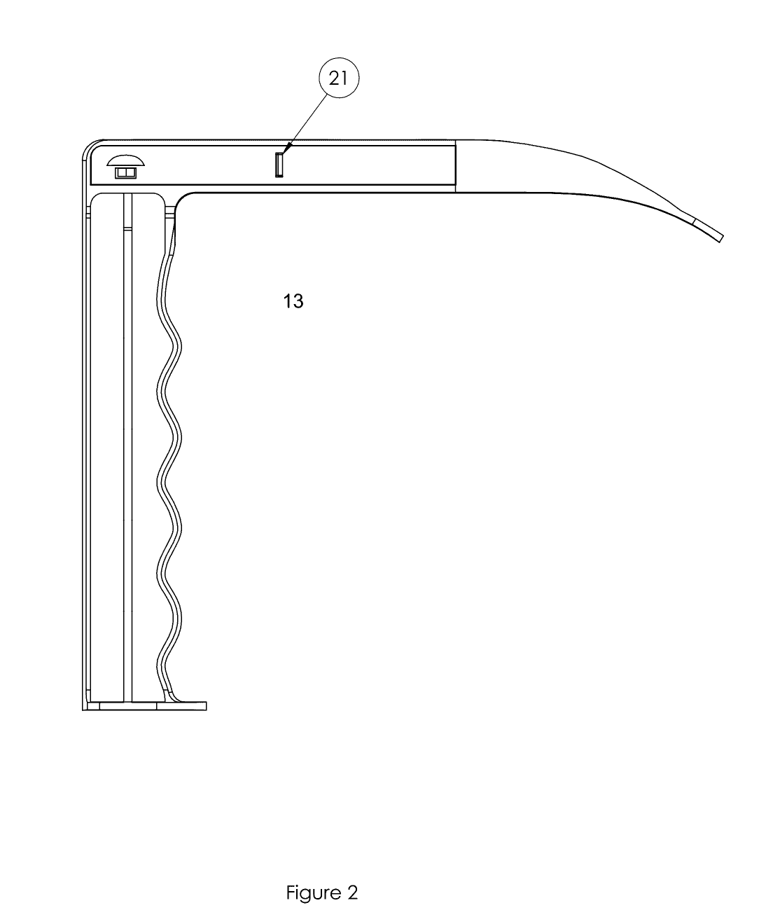

As shown in FIG. 2, a snap latch 21 snaps cover 14 into place. Snap latch 21 attaches cover 14 firmly into place over housing cavity 13. During extensive use of the laryngoscope 10, snap latch 21 ensures that cover 14 remains securely in place over the housing cavity 13 when stress is placed on cover 14. The laryngoscope 10 may include one snap latch 21, or may include a plurality of snap latches 21 to ensure that cover 14 remains tightly secured over blade 12.

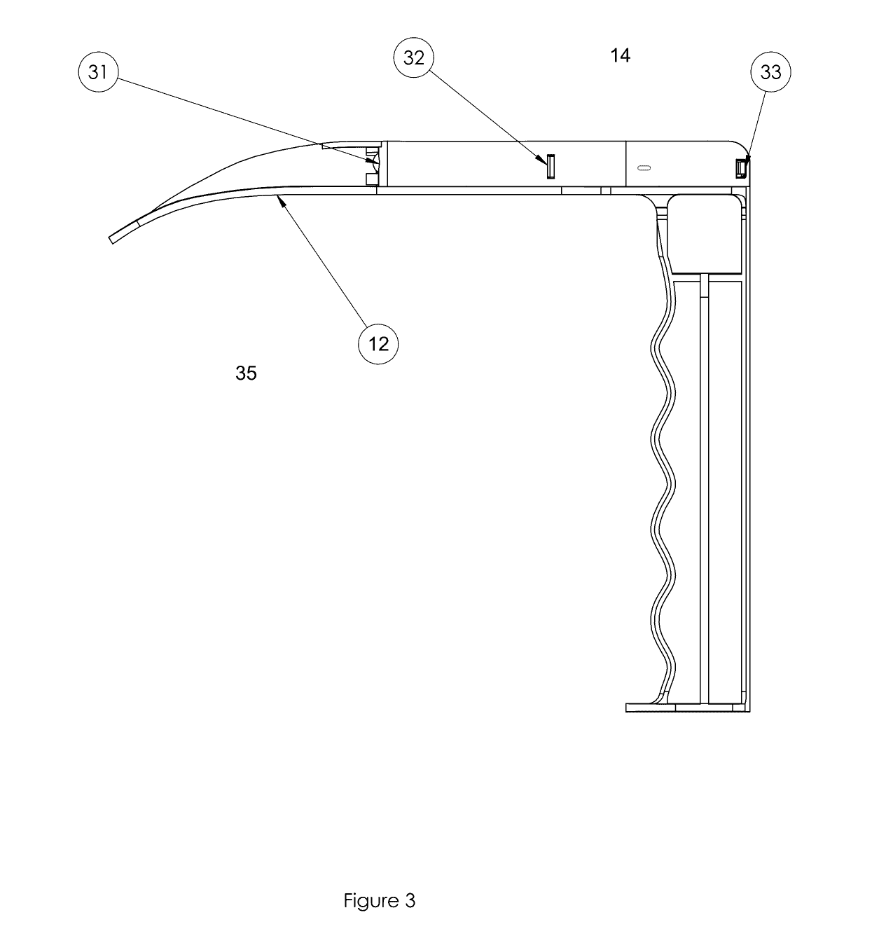

As illustrated in FIG. 3, housing cavity 13 includes light source 31, such as an LED bulb, protruding from a distal end of the housing cavity 13. Light source 31 is arranged such that it projects light toward the distal end 35 of the blade 12.

Blade 12 includes a proximal portion that is substantially flat. The proximal portion is located closer to the handle 11. A second portion of the blade 12, located toward the distal end of the blade 12, is a curved portion.

In an exemplary embodiment, the blade b 12 with a flat proximal portion and a curved distal portion is a Miller-style blade. In another exemplary embodiment, the blade 12 is manufactured in accordance with the “Mac” or “Macintosh” style blades, which includes a continuous curve, without a flat portion, from the proximal end of the blade 12 to the distal end of the blade 12.

Cover 14 may be secured over the housing cavity 13 located on blade 12, using snap fittings 32 and 33.

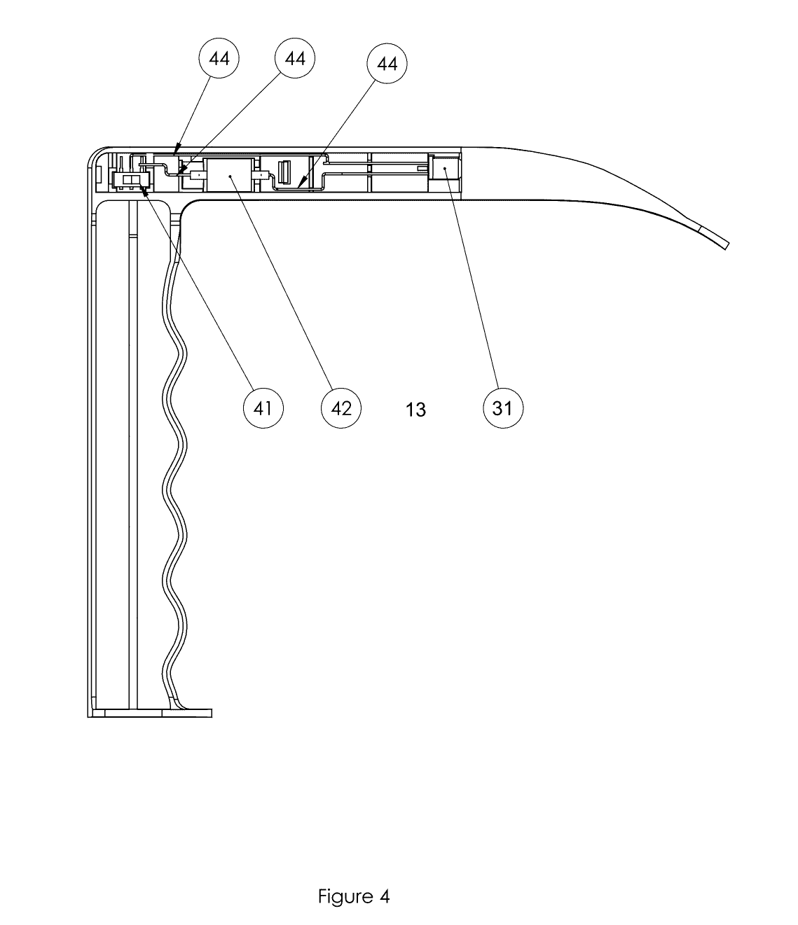

FIG. 4 illustrates a side view, such as a right side view, of an exemplary embodiment of the laryngoscope 10 with cover 14 removed from the housing cavity 13. Shown is the interior of housing cavity 13. Light source 31 is connected via a series of interconnecting wires 44 to a battery pack 42. The wires 44 connect battery pack 42 to a switch 41. Light source 31, battery pack 42, switch 41 and interconnecting wires 44 form a circuit that can be energized by the flipping of switch 41 to either activate or deactivate light source 31.

As shown, battery pack 42 is arranged toward the proximal end of blade 12. In another embodiment, battery pack 42 may be located closer to the proximal end of the blade 12. Due to the placement of battery pack 42 toward the proximal end, and due to the placement of battery pack 42 relative to light source 31, the housing cavity 13 is of a small size, and is sized to taper in size such that it is smaller at a distal end of blade 12. Thus, housing cavity 13 tapers to gradually reduce in size as it proceeds from the proximal end to the distal end of the blade 12.

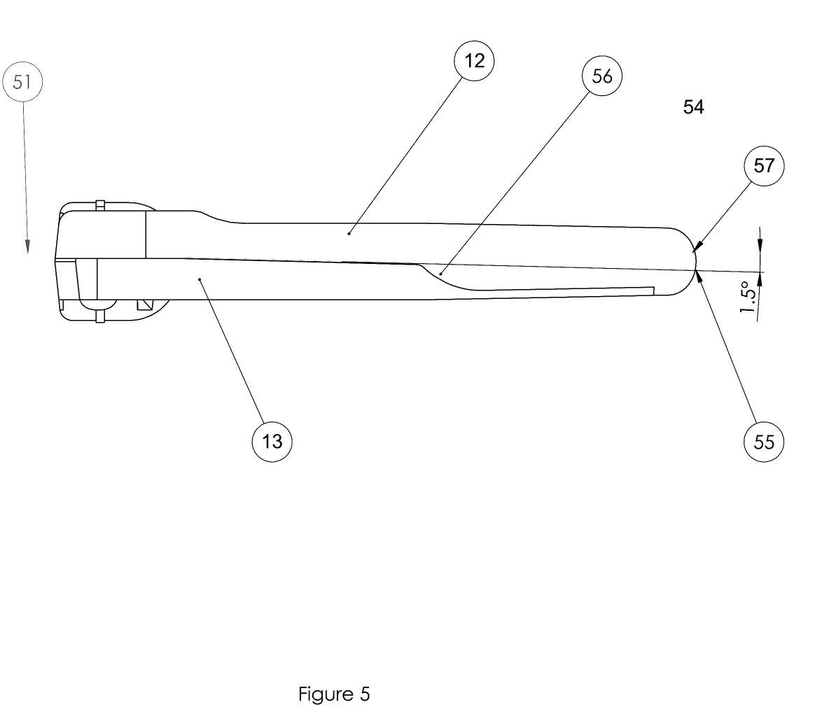

FIG. 5 illustrates a top-down view of an exemplary embodiment. In an embodiment, a medical professional looks down the length of blade 12 from the proximal end 51, toward the distal end 54. In an embodiment, a medical professional uses blade 12 to displace a patient's tongue and other soft tissue, in order to visualize the vocal cords. Light emanates from light source 31, located in area 56, and illuminates the distal end 54 of the blade 12.

In an exemplary embodiment, housing cavity 13 tapers as it travels from the proximal end 51 to the distal end 54. The tapering of the housing cavity 13 is at angle of 1.5 degree, or an approximate angle of 1.5 degrees. In another embodiment, the tapering angle is any additional suitable angle, such as 1 degree, 2 degrees, or any other suitable taper angle.

Based on the tapering of the housing cavity 13, the visualization area at the distal end 54 of the blade is increased such that the area between location 57 and location 55, which would have been outside of the original visualization area without a tapering of the housing cavity 13, is now within the visualization area. Thus, in this exemplary embodiment, the visualization area is increased from approximately half the blade tip width to three-quarters of the blade tip width (the increase in width includes the tip area from location 54 to location 57, which is approximately one-quarter of the blade tip width). Thus an increase of one-quarter of the blade tip width occurs due to the tapering, resulting in a 50 % increase in the visualization. As a result, the intubation success rate increases, due to the increase in visualization area.

One or more exemplary aspects comprise a laryngoscope assembly comprising: (a) a handle; (b) a blade, the blade including a cavity, the cavity housing a lighting system; and (c) a cover locked in place over the cavity via a snap-latch, wherein the cavity is sized such that it tapers in size from a proximal end of the blade to a distal end of the blade.

In one or more exemplary embodiments: (1) the laryngoscope further comprises an activation mechanism, the activation mechanism including a switch; (2) the laryngoscope further comprises an activation mechanism including an insulating tab that projects outward from the laryngoscope, wherein upon removal of the insulating tab a light source is activated; (3) the blade is formed substantially straight, in a style of a Miller blade; and/or (4) the blade is formed substantially curved, in a style of a Macintosh blade.

Another aspect may comprise a laryngoscope assembly comprising: (a) a handle; (b) a blade extending from the handle, the blade including a cavity; and (c) a lighting system housed within the cavity and including a light source, power source, activation device, and switch; wherein the cavity is sized such that it tapers in size from a proximal end of the blade to a distal end of the blade.

Laryngoscopes of one or more exemplary embodiments may be sterilized after manufacture and dispatched in sterile packaging for single-use.

In one or more embodiments, the blade and the handle (referred to herein collectively as “the body”) are integrally molded. In at least one exemplary embodiment, the material of which the body is formed is a strong, rigid, lightweight plastic (e.g., a polymer).

One example of a suitable plastic is a glass-fiber reinforced polyarylamide compound that provides high strength and rigidity, surface gloss, and creep resistance. An exemplary embodiment uses a 50% glass-fiber reinforced polyarylamide compound, but those skilled in the art will understand that other percentages may be used without departing from the spirit and scope of the claimed invention.

Polyarylamides are thermoplastic crystalline polymers of aromatic diamines and aromatic dicarboxylic anhydrides having good heat, fire, and chemical resistance, property retention at high temperatures, dielectric and mechanical properties, and stiffness but low light resistance and processability. Those skilled in the art will understand that other plastics with suitable strength and rigidity also may be used.

In one or more embodiments, the body is made of a plastic (such as glass-fiber reinforced polyarylamide) having properties of at least one of radiolucence and non-conductivity. As used herein, “radiolucence ” means high transparency to radiation, so that the device may be used when taking, for example, x-ray images. “Nonconductive, ” as used herein, means essentially dielectric.

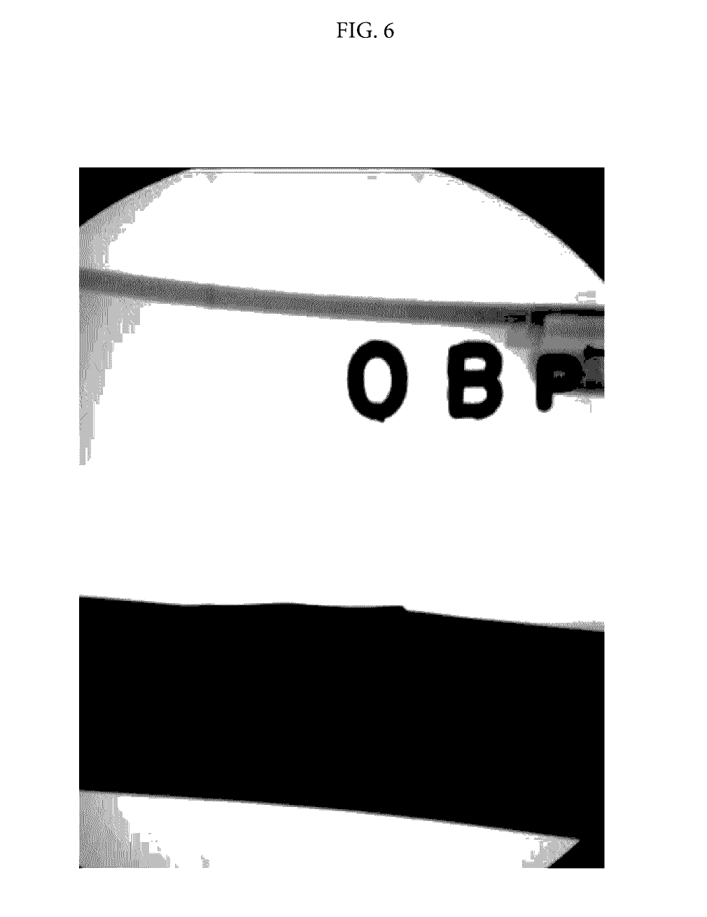

An advantage of radiolucence is that the device may be used when taking X-ray images, without obscuring essential structures, as shown in FIG. 6. The “OBP” in FIG. 6 resulted from metal lettering placed below the blades of an embodiment to show the radiolucency. The much darker image on the left is of a stainless steel comparison blade, which shows up as black due to its opacity with respect to X-rays.

Embodiments described herein may provide light to the tip of the laryngoscope and still remain highly (as much as 99%) radiolucent. Prior art devices have, for example, fiber optic cables that obstruct the view when X-ray images are taken, even when the devices are constructed of plastic. Metal devices are, of course, not radiolucent at all.

This radiolucent property means that laryngoscopes described herein may not need to be removed prior to the use of imaging techniques in surgical procedures. This can expedite the conduct of a procedure needing anatomic identification and/or device localization.

An advantage of nonconductivity is that it provides improved safety to patients—in contrast to metal laryngoscopes. Currents as low as 0.001A may be felt by a patient, and larger currents may damage the patient. Embodiments described herein limit currents to less than 10⁻⁶ A, and thus greatly reduce electrical hazards.

For example, electro-cautery is used extensively in surgical tissue dissection. The use of metal laryngoscopes exposes the operating surgeon and the patient to the risk of retracted tissue damage due to destructive cautery current being conducted inadvertently. Laryngoscopes are often used to displace and retract delicate cautery sensitive tissues. Cautery injury to these tissues can create major complications. Use of a non-electrical conducting material, such as is described herein with respect to certain embodiments, prevents any stray electrical energy injury to the retracted tissues. Patient safety is thus enhanced.

As those skilled in the art will understand, strength is a function of both the material and the design. Designs using weaker material than is described herein need to be thicker and more rounded. Both of these traits will decrease the favorability of a laryngoscope, which should not block visibility of the body part.

Flexural Strength represents the limit before a material will break under stress. Flexural modulus is the tendency of the material to bend under stress. Both of these parameters are critical to laryngoscope design and resulting performance. First, a laryngoscope blade must be thin enough to not interfere with the medical procedure for which it is used. Very thick blades will tend to fill the space that the physician needs to work in. An optimal design will have a blade thin enough to allow space for the physician to work. Typically metal blades are used because of their high Flexural modulus. They have very high flexural strength, because they bend rather than break. Metal blades as thin as 0.5-2.0 mm are readily available and this thickness is small enough to not interfere with the physician's work space in a wound or operating cavity. Stainless steel metal can have a flexural modulus of 180 Gpa which will inhibit blade deformation of more than 10 mm under 15 lbs of tip pressure for most retractor designs.

Plastic injection molded blades require a thicker blade because they have a lower Flexural Modulus. Blade strength will increase as the cube of the blade thickness, but blade thicknesses larger than 2 mm are not desirable in most physician applications.

Typical plastic materials, such as those shown in Table 1 below, have a Flexural Modulus of just a few Gpa and a Flexural Strength of less than 200 Mpa. These lower value parameters result in laryngoscope blades that deform more than 10 mm under use, and are likely to break with less than 30 lbs of force placed on the tip of an average length laryngoscope blade (50-150 mm long).

Laryngoscope blades that deform significantly during use increase the physician's difficulty in retracting the tissue during a medical procedure. Laryngoscope blades that break with less than 30 lbs of force can create a hazard to the patient since a broken blade, or pieces of a broken blade, may fall into the patient and create damage. Laryngoscope blades made from the plastics listed in the following table will typically bend more than 20 mm under 10 lbs of tip force, and will break at 15 lbs (or even less) of tip force.

TABLE 1
TYPICAL FLEXURAL STRENGTH AND
FLEXURAL MODULUS OF POLYMERS

| | FLEXURAL | FLEXURAL |
|---|---|---|
| | STRENGTH | STRENGTH |
| POLYMER TYPE | (MPa) | (MPa) |
| Polyamide-lmide | 175 | 5 |
| Polycarbonate | 90 | 2.3 |
| Polyethylene, MDPE | 40 | 0.7 |
| Polyethylene Terephthalate | 80 | 1 |
| (PET) | | |

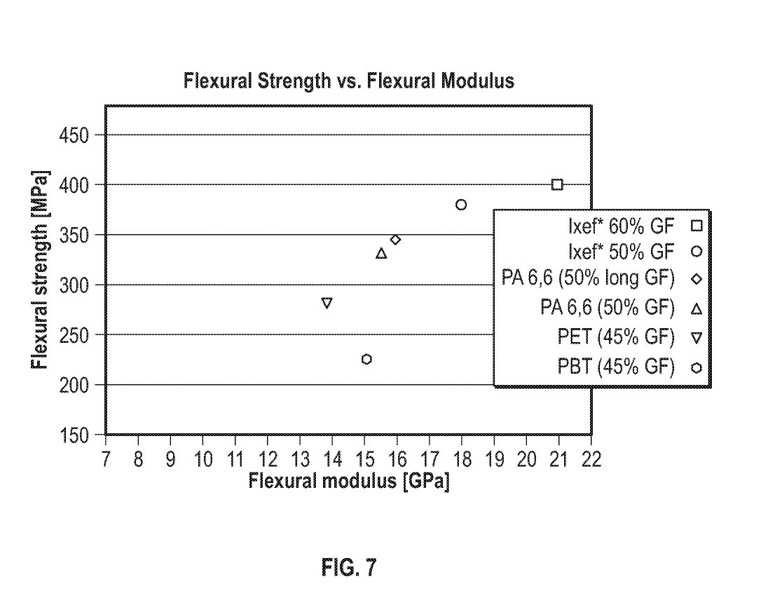

To increase the flexural modulus and flexural strength of plastic, in an embodiment, glass fiber is added to the plastic material. FIG. 7 shows a variety of plastics with various percentages of glass fiber added.

It can be seen from the above that the addition of glass fiber can increase the Flexural Strength of certain plastics to 300 Mpa or above, and increase the Flexural Modulus to 16 Gpa or above. In an exemplary embodiment, a certain type of plastic, polyarylamide, is infused with glass fiber to create a flexural strength of over 375 Gpa and a Flexural modulus of over 17 Gpa.

Plastics with these properties have the ability to create laryngoscope blades of approximately 2 mm thickness that withstand over 30 lbs of tip force without breaking and deform less than 10 mm under 15 lbs of force. Additionally, the glass fiber in this material will “glassify” at the surface leaving a very smooth “metal like” finish which is highly desirable in laryngoscope applications.

The glass fiber in the material also will decrease the likelihood of sharp shards of material being created during an overstress and breakage event. This tendency to create dull edges upon breakage decreases the likelihood that a patient will experience damage if the laryngoscope is overstressed and ultimately broken.

Additionally, the way in which a material breaks can be important in medical applications. The breakage characteristics of a material are often measured by Impact Strength. Materials with low impact strength (10-20 J/M) can break under stress into large numbers of sharp shards which can pose a hazard to a patient if material failure occurs during a medical procedure. Sharp shards can cut patient tissue and large numbers of these shards can make it difficult or impossible to remove the broken material from the patient.

Materials (such as glass fiber reinforced polyarylamide) used in certain embodiments described herein have a high impact strength (>100 J/M) and will fail with very few fractured component edges (and the resulting edges will be blunt). This breakage characteristic minimizes potential hazard to a patient during product overstress that results in material breakage.

It should be understood that the foregoing relates to exemplary embodiments of the invention and that modifications may be made without departing from the spirit and scope of the claimed invention.

We claim:

1. A laryngoscope assembly comprising:
a handle;
a blade, the blade including a cavity, the cavity housing a lighting system; and
a cover locked in place over the cavity via a snap-latch,
wherein at least the blade is molded from a semi-crystalline polymer.

2. An assembly as in claim 1, wherein the blade further comprises an activation mechanism, the activation mechanism including a switch.

3. An assembly as in claim 1, wherein the cavity is sized such that it tapers in size from a proximal end of the blade to a distal end of the blade.

4. An assembly as in claim 1, wherein the blade further comprises an activation mechanism including an insulating tab that projects outward from the blade, and wherein upon removal of the insulating tab a light source is activated.

5. An assembly as in claim 1, wherein the blade is formed substantially straight, in a style of a Miller blade.

6. An assembly as in claim 1, wherein the blade is formed substantially curved, in a style of a Macintosh blade.

7. A laryngoscope assembly comprising:
a handle;
a blade extending from the handle, the blade including a cavity; and
a lighting system housed within the cavity and including a light source, power source, activation device, and switch, wherein at least the blade is molded from a semi crystalline polymer.

8. An assembly as in claim 7, wherein the switch further comprises an activation mechanism including an insulating tab that projects outward from the blade, and wherein upon removal of the insulating tab a light source is activated.

9. An assembly as in claim 7, wherein the cavity is sized such that it tapers in size from a proximal end of the blade to a distal end of the blade.

10. An assembly as in claim 7, wherein the blade is formed substantially straight, in a style of a Miller blade.

11. An assembly as in claim 7, wherein the blade is formed substantially curved, in a style of a Macintosh blade.

12. An assembly as in claim 7, wherein the light source is an LED light source.

13. An assembly as in claim 1, wherein at least the blade is molded from a low conductivity polymer.

14. An assembly as in claim 1, wherein at least the blade is molded from a radiolucent polymer.

15. An assembly as in claim 1, wherein at least the blade is molded from a polymer that is at least 50% glass-fiber reinforced.

16. An assembly as in claim 1, wherein at least the blade is molded from a polymer that is a polyarylamide compound.

17. An assembly as in claim 1, wherein at least the blade is molded from a thermoplastic crystalline polymer.

18. An assembly as in claim 1, wherein at least the blade is molded from a thermoplastic crystalline polymer of aromatic diamines and aromatic dicarboxylic anhydrides.

19. An assembly as in claim 1, wherein at least the blade is molded from an at least 50% glass-fiber reinforced polyarylamide.

20. An assembly as in claim 1, wherein at least the blade is molded from a polymer with a conductivity of less than 10⁻⁶A.

21. An assembly as in claim 1, wherein at least the blade is molded from a polymer with a flexural modulus of at least 17 Gpa.

22. An assembly as in claim 1, wherein at least the blade is molded from a polymer with a flexural strength of at least 375 Mpa.

23. An assembly as in claim 1, wherein at least the blade is molded from a polymer with an impact strength of at least 100 J/M.

24. A laryngoscope assembly comprising:
a handle;
a blade extending from the handle, the blade including a cavity; and
a lighting system housed within the cavity and including a light source, power source, activation device, and switch, wherein at least the blade is molded from polyarylamide.

25. An assembly as in claim 24, wherein the cavity is sized such that it tapers in size from a proximal end of the blade to a distal end of the blade.
